(19) United States
(12) Patent Application Publication
(10) Pub. No.: US 2019/0194387 A1
(43) Pub. Date: Jun. 27, 2019

(54) METHOD OF PREPARING GLYCOLIDE

(71) Applicant: Nanjing University, Nanjing (CN)

(72) Inventors: Hong LI, Nanjing (CN); Quanxing ZHANG, Nanjing (CN); Jiaye SHENG, Nanjing (CN); Wei HUANG, Nanjing (CN); Wei JIANG, Nanjing (CN); Aimin LI, Nanjing (CN); Bingcai PAN, Nanjing (CN)

(21) Appl. No.: 16/293,595

(22) Filed: Mar. 5, 2019

Related U.S. Application Data
(63) Continuation-in-part of application No. PCT/CN2017/100510, filed on Sep. 5, 2017.

(30) Foreign Application Priority Data
Sep. 7, 2016 (CN) 201610809645.9

(51) Int. Cl. C08G 63/08; C08G 63/82; C07D 319/12; B01J 31/02

(52) U.S. Cl. CPC C08G 63/08; C08G 63/82; B01J 31/0244; B01J 31/0251; C07D 319/12

(57) ABSTRACT

A method of preparing glycolide, the method including dehydrative oligomerization and catalytic depolymerization. The dehydrative oligomerization includes stepwise heating a glycolic acid aqueous solution from room temperature to a temperature of between 200 and 210° C., and maintaining the temperature at each corresponding temperature stage, to yield an oligoglycolic acid. The catalytic depolymerization includes adding a binary complex catalytic system to the oligoglycolic acid to yield a reactant mixture, stepwise heating the reactant mixture from room temperature to a temperature of between 280 and 285° C., and maintaining the temperature at each corresponding temperature stage.

### CROSS-REFERENCE TO RELAYED APPLICATIONS

This application is a continuation-in-part of International Patent Application No. PCT/CN2017/100510 with an international filing date of Sep. 5, 2017, designating the United States, now pending, and further claims foreign priority benefits to Chinese Patent Application No. 201610809645.9 filed Sep. 7, 2016. The contents of all of the aforementioned applications, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, Mass. 02142.

### BACKGROUND

This disclosure relates to a method of preparing glycolide.

Conventional synthesis of glycolide includes catalytic depolymerization performed with a high temperature (220-250° C.) or in a high boiling solvent. However, it is difficult to separate the high boiling solvent from the product and to recycle it. In addition, conventional catalysts for the synthesis of glycolide are metal compounds, such as Sn(Oct)₂, SnCl₂, Sb₂O₃. They are cytotoxic and difficult to recycle.

### SUMMARY

Disclosed is a method of preparing glycolide which is safe, environmentally friendly; the glycolide is prepared in a high yield and is of high purity.

Disclosed is a method of preparing glycolide, the method comprising dehydrative oligomerization and catalytic depolymerization, the dehydrative oligomerization comprising:
a1) adding a 70 wt. % glycolic acid aqueous solution to a reactor, heating the glycolic acid aqueous solution to a temperature of between 100 and 110° C. at a heating rate of 1.0-1.5° C./min, and maintaining the temperature under atmosphere for 1.0-2.0 hours;
b1) heating the glycolic acid aqueous solution from the temperature of between 100 and 110° C. to a temperature of between 140 and 150° C. at a heating rate of 1.0-1.5° C./min, and maintaining the temperature under atmosphere for 1.0-2.0 hours;
c1) heating the glycolic acid aqueous solution from the temperature of between 140 and 150° C. to a temperature of between 155 and 165° C. at a heating rate of 1.0-1.5° C./min, and maintaining the temperature under atmosphere for 1.0-2.0 hours;
d1) heating the glycolic acid aqueous solution from the temperature of between 155 and 165° C. to a temperature of between 200 and 210° C. at a heating rate of 1.0-1.5° C./min, decreasing a pressure of the glycolic acid aqueous solution from the atmosphere to an absolute pressure of between 90.0 and 95.0 torr at a decompression rate of 1.0-1.5 torr/min, and maintaining the temperature and pressure for 1.0-1.5 hours;
e1) under the temperature of between 200 and 210° C., decreasing the pressure of the glycolic acid aqueous solution from the absolute pressure of between 90.0 and 95.0 torr to an absolute pressure of between 10.0 and 20.0 torr at a decompression rate of 1.0-1.5 torr/min, and maintaining the temperature and pressure for 1.0-2.0 hours, to yield an oligoglycolic acid having a weight-average molecular weight (M_{w}) of 5.0-6.0×10³, and a yield of 100%; and

the catalytic depolymerization comprising:
a2) adding a binary complex catalytic system to the oligoglycolic acid obtained in e1), to yield a reactant mixture;
b2) reducing an absolute pressure of the reactant mixture from atmosphere to between 3.0 and 5.0 torr at a decompression rate of 1.0-1.5 torr/min;
c2) heating the reactant mixture to a temperature of between 250 and 260° C. at a heating rate of 10-15° C./min, and maintaining the temperature for 20-30 minutes;
d2) heating the reactant mixture from the temperature of between 250 and 260° C. to a temperature of between 270 and 275° C. at a heating rate of 1.0-1.5° C./min, and maintaining the temperature for 20-30 minutes; and

e2) heating the reactant mixture from the temperature of between 270 and 275° C. to a temperature of between 280 and 285° C. at a heating rate of 1.0-1.5° C./min, and maintaining the temperature for 60-70 minutes, to yield a white or yellowish crude glycolide having a purity greater than or equal to 99.9%, and a yield greater than or equal to 98.0%.

The binary complex catalytic system can comprise a cyclic guanidine compound and a linear guanidine compound; the cyclic guanidine compound can be selected from bicyclic guanidine, guanine, and benzimidazole; the linear guanidine compound can be selected from glycocyamine, creatine, and phosphocreatine, and an addition amount of each of the cyclic guanidine compound and the linear guanidine compound can account for 0.05-0.10 wt. % of that of the oligoglycolic acid.

The oligoglycolic acid obtained in e1) can have a weight-average molecular weight (M_{w}) of 5.0-6.0×10³, and is prepared in a yield of 100%.

The crude glycolide obtained in e2) can be white or yellowish and can have a purity greater than or equal to 99.9%, and is prepared in a yield greater than or equal to 98.0%.

Advantages of the method of preparing glycolide as described in the disclosure are summarized as follows.

1. The binary complex catalytic system is a non-cytotoxic guanidine compound with relatively high catalytic efficiency and relatively low addition amount.

2. The glycolide is prepared in high yield (≥98.0%) and is of high purity (≥99.9%).

3. The method is an efficient, environmentally friendly, closed cycle process.

### DETAILED DESCRIPTION

To further illustrate, embodiments detailing a method of preparing glycolide are described below. It should be noted that the following embodiments are intended to describe and not to limit the disclosure.

### Example 1

50 kg of a 70 wt. % glycolic acid aqueous solution was added to a reactor for dehydrative oligomerization. First, the glycolic acid aqueous solution was heated from room temperature to 100° C. at a heating rate of 1.0° C./min, and then dehydrated under atmosphere for 1.0 hour. Second, the glycolic acid aqueous solution was heated from 100° C. to 140° C. at a heating rate of 1.0° C./min, and then dehydrated under atmosphere for 1.0 hour. Third, the glycolic acid aqueous solution was further heated from 140° C. to 155° C. at a heating rate of 1.0° C./min, and then dehydrated under atmosphere for 1.0 hour. Fourth, the glycolic acid aqueous solution was further heated from 155° C. to 200° C. at a heating rate of 1.0° C./min, and the absolute pressure of the glycolic acid aqueous solution was reduced from atmosphere to 90.0 torr at a decompression rate of 1.0 torr/min. Hold the temperature and the pressure for an hour. Thereafter, the absolute pressure of the glycolic acid aqueous solution was reduced from 90.0 torr to 10.0 torr at a decompression rate of 1.0 torr/min for an hour. A product of oligoglycolic acid having a weight-average molecular weight (M_{w}) of 5.0×10³ was obtained, and a yield thereof was 100%.

25.0 g of bicyclic guanidine and 25.0 g of glycocyamine were added to the product of oligoglycolic acid. The absolute pressure of the reactant mixture was reduced from atmosphere to 3.0 torr at a decompression rate of 1.0 torr/min. Under the pressure, the reactant mixture was heated to 250° C. at a heating rate of 10° C./min, and the temperature was held for 20 minutes. Thereafter, the reactant mixture was heated from 250° C. to 270° C. at a heating rate of 1.0° C./min, and the temperature was held for 20 minutes. Next, the reactant mixture was further heated from 270° C. to 280° C. at a heating rate of 1.0° C./min, and the temperature was held for 60 minutes, to yield a white or yellowish crude glycolide.

The crude glycolide was washed using a 0.1 wt. % alkali solution and deionized water to be neutral, and then vacuum dried at 20° C. for 24 hours. The obtained glycolide has a yield of 98.5% and a purity of 99.9%.

### Comparison Example 1

The preparation method is basically the same as that in Example 1 except that only 25.0 g of bicyclic guanidine was added in the catalytic depolymerization, and the obtained glycolide has a yield of 41.1% and a purity of 82.5%.

### Comparison Example 2

The preparation method is basically the same as that in Example 1 except that only 25.0 g of glycocyamine was added in the catalytic depolymerization, and the obtained glycolide has a yield of 34.5% and a purity of 85.3%.

### Example 2

50 kg of a 70 wt. % glycolic acid aqueous solution was added to a reactor for dehydrative oligomerization. First, the glycolic acid aqueous solution was heated from room temperature to 100° C. at a heating rate of 1.0° C./min, and then dehydrated under atmosphere for 1.0 hour. Second, the glycolic acid aqueous solution was heated from 100° C. to 140° C. at a heating rate of 1.0° C./min, and then dehydrated under atmosphere for 1.0 hour. Third, the glycolic acid aqueous solution was further heated from 140° C. to 155° C. at a heating rate of 1.0° C./min, and then dehydrated under atmosphere for 1.0 hour. Fourth, the glycolic acid aqueous solution was further heated from 155° C. to 200° C. at a heating rate of 1.0° C./min, and the absolute pressure of the glycolic acid aqueous solution was reduced from atmosphere to 90.0 torr at a decompression rate of 1.0 torr/min. Hold the temperature and the pressure for an hour. Thereafter, the absolute pressure of the glycolic acid aqueous solution was reduced from 90.0 torr to 10.0 torr at a decompression rate of 1.0 torr/min for an hour. A product of oligoglycolic acid having a weight-average molecular weight (M_{w}) of 5.0×10³ was obtained, and a yield thereof was 100%.

50.0 g of bicyclic guanidine and 35.0 g of creatine were added to the product of oligoglycolic acid. The absolute pressure of the reactant mixture was reduced from atmosphere to 3.0 torr at a decompression rate of 1.0 torr/min. Under the pressure, the reactant mixture was heated to 250° C. at a heating rate of 10° C./min, and the temperature was held for 20 minutes. Thereafter, the reactant mixture was heated from 250° C. to 270° C. at a heating rate of 1.0° C./min, and the temperature was held for 20 minutes. Next, the reactant mixture was further heated from 270° C. to 280° C. at a heating rate of 1.0° C./min, and the temperature was held for 60 minutes, to yield a white or yellowish crude glycolide.

The crude glycolide was washed using a 0.1 wt. % alkali solution and deionized water to be neutral, and then vacuum dried at 20° C. for 24 hours. The obtained glycolide has a yield of 98.2% and a purity of 99.9%.

### Example 3

50 kg of a 70 wt. % glycolic acid aqueous solution was added to a reactor for dehydrative oligomerization. First, the glycolic acid aqueous solution was heated from room temperature to 100° C. at a heating rate of 1.0° C./min, and then dehydrated under atmosphere for 1.0 hour. Second, the glycolic acid aqueous solution was heated from 100° C. to 140° C. at a heating rate of 1.0° C./min, and then dehydrated under atmosphere for 1.0 hour. Third, the glycolic acid aqueous solution was further heated from 140° C. to 155° C. at a heating rate of 1.0° C./min, and then dehydrated under atmosphere for 1.0 hour. Fourth, the glycolic acid aqueous solution was further heated from 155° C. to 200° C. at a heating rate of 1.0° C./min, and the absolute pressure of the glycolic acid aqueous solution was reduced from atmosphere to 90.0 torr at a decompression rate of 1.0 torr/min. Hold the temperature and the pressure for an hour. Thereafter, the absolute pressure of the glycolic acid aqueous solution was reduced from 90.0 torr to 10.0 torr at a decompression rate of 1.0 torr/min for an hour. A product of oligoglycolic acid having a weight-average molecular weight (M_{w}) of 5.0×10³ was obtained, and a yield thereof was 100%.

35.0 g of bicyclic guanidine and 50.0 g of phosphocreatine were added to the product of oligoglycolic acid. The absolute pressure of the reactant mixture was reduced from atmosphere to 3.0 torr at a decompression rate of 1.0 torr/min. Under the pressure, the reactant mixture was heated to 250° C. at a heating rate of 10° C./min, and the temperature was held for 20 minutes. Thereafter, the reactant mixture was heated from 250° C. to 270° C. at a heating rate of 1.0° C./min, and the temperature was held for 20 minutes. Next, the reactant mixture was further heated from 270° C. to 280° C. at a heating rate of 1.0° C./min, and the temperature was held for 60 minutes, to yield a white or yellowish crude glycolide.

The crude glycolide was washed using a 0.1 wt. % alkali solution and deionized water to be neutral, and then vacuum dried at 20° C. for 24 hours. The obtained glycolide has a yield of 98.7% and a purity of 99.9%.

Example 4

100 kg of a 70 wt. % glycolic acid aqueous solution was added to a reactor for dehydrative oligomerization. First, the glycolic acid aqueous solution was heated from room temperature to 105° C. at a heating rate of 1.2° C./min, and then dehydrated under atmosphere for 1.2 hour. Second, the glycolic acid aqueous solution was heated from 105° C. to 145° C. at a heating rate of 1.2° C./min, and then dehydrated under atmosphere for 1.2 hour. Third, the glycolic acid aqueous solution was further heated from 145° C. to 160° C. at a heating rate of 1.0° C./min, and then dehydrated under atmosphere for 1.2 hour. Fourth, the glycolic acid aqueous solution was further heated from 160° C. to 205° C. at a heating rate of 1.2° C./min, and the absolute pressure of the glycolic acid aqueous solution was reduced from atmosphere to 92.0 torr at a decompression rate of 1.2 torr/min. Hold the temperature and the pressure for 1.2 hour. Thereafter, the absolute pressure of the glycolic acid aqueous solution was reduced from 92.0 torr to 15.0 torr at a decompression rate of 1.2 torr/min for 1.5 hour. A product of oligoglycolic acid having a weight-average molecular weight (M_{w}) of 5.5×10³ was obtained, and a yield thereof was 100%.

100.0 g of guanine and 70.0 g of glycocyamine were added to the product of oligoglycolic acid. The absolute pressure of the reactant mixture was reduced from atmosphere to 4.0 torr at a decompression rate of 1.2 torr/min. Under the pressure, the reactant mixture was heated to 255° C. at a heating rate of 12° C./min, and the temperature was held for 25 minutes. Thereafter, the reactant mixture was heated from 255° C. to 272° C. at a heating rate of 1.2° C./min, and the temperature was held for 25 minutes. Next, the reactant mixture was further heated from 272° C. to 282° C. at a heating rate of 1.2° C./min, and the temperature was held for 65 minutes, to yield a white or yellowish crude glycolide.

The crude glycolide was washed using a 0.1 wt. % alkali solution and deionized water to be neutral, and then vacuum dried at 20° C. for 24 hours. The obtained glycolide has a yield of 98.6% and a purity of 99.9%.

### Example 5

100 kg of a 70 wt. % glycolic acid aqueous solution was added to a reactor for dehydrative oligomerization. First, the glycolic acid aqueous solution was heated from room temperature to 105° C. at a heating rate of 1.2° C./min, and then dehydrated under atmosphere for 1.2 hour. Second, the glycolic acid aqueous solution was heated from 105° C. to 145° C. at a heating rate of 1.2° C./min, and then dehydrated under atmosphere for 1.2 hour. Third, the glycolic acid aqueous solution was further heated from 145° C. to 160° C. at a heating rate of 1.0° C./min, and then dehydrated under atmosphere for 1.2 hour. Fourth, the glycolic acid aqueous solution was further heated from 160° C. to 205° C. at a heating rate of 1.2° C./min, and the absolute pressure of the glycolic acid aqueous solution was reduced from atmosphere to 92.0 torr at a decompression rate of 1.2 torr/min. Hold the temperature and the pressure for 1.2 hour. Thereafter, the absolute pressure of the glycolic acid aqueous solution was reduced from 92.0 torr to 15.0 torr at a decompression rate of 1.2 torr/min for 1.5 hour. A product of oligoglycolic acid having a weight-average molecular weight (M_{w}) of 5.5×10³ was obtained, and a yield thereof was 100%.

50.0 g of guanine and 50.0 g of creatine were added to the product of oligoglycolic acid. The absolute pressure of the reactant mixture was reduced from atmosphere to 4.0 torr at a decompression rate of 1.2 torr/min. Under the pressure, the reactant mixture was heated to 255° C. at a heating rate of 12° C./min, and the temperature was held for 25 minutes. Thereafter, the reactant mixture was heated from 255° C. to 272° C. at a heating rate of 1.2° C./min, and the temperature was held for 25 minutes. Next, the reactant mixture was further heated from 272° C. to 282° C. at a heating rate of 1.2° C./min, and the temperature was held for 65 minutes, to yield a white or yellowish crude glycolide.

The crude glycolide was washed using a 0.1 wt. % alkali solution and deionized water to be neutral, and then vacuum dried at 20° C. for 24 hours. The obtained glycolide has a yield of 98.3% and a purity of 99.9%.

### Example 6

100 kg of a 70 wt. % glycolic acid aqueous solution was added to a reactor for dehydrative oligomerization. First, the glycolic acid aqueous solution was heated from room temperature to 105° C. at a heating rate of 1.2° C./min, and then dehydrated under atmosphere for 1.2 hour. Second, the glycolic acid aqueous solution was heated from 105° C. to 145° C. at a heating rate of 1.2° C./min, and then dehydrated under atmosphere for 1.2 hour. Third, the glycolic acid aqueous solution was further heated from 145° C. to 160° C. at a heating rate of 1.0° C./min, and then dehydrated under atmosphere for 1.2 hour. Fourth, the glycolic acid aqueous solution was further heated from 160° C. to 205° C. at a heating rate of 1.2° C./min, and the absolute pressure of the glycolic acid aqueous solution was reduced from atmosphere to 92.0 torr at a decompression rate of 1.2 torr/min. Hold the temperature and the pressure for 1.2 hour. Thereafter, the absolute pressure of the glycolic acid aqueous solution was reduced from 92.0 torr to 15.0 torr at a decompression rate of 1.2 torr/min for 1.5 hour. A product of oligoglycolic acid having a weight-average molecular weight (M_{w}) of 5.5×10³ was obtained, and a yield thereof was 100%.

70.0 g of guanine and 100.0 g of phosphocreatine were added to the product of oligoglycolic acid. The absolute pressure of the reactant mixture was reduced from atmosphere to 4.0 torr at a decompression rate of 1.2 torr/min. Under the pressure, the reactant mixture was heated to 255° C. at a heating rate of 12° C./min, and the temperature was held for 25 minutes. Thereafter, the reactant mixture was heated from 255° C. to 272° C. at a heating rate of 1.2° C./min, and the temperature was held for 25 minutes. Next, the reactant mixture was further heated from 272° C. to 282° C. at a heating rate of 1.2° C./min, and the temperature was held for 65 minutes, to yield a white or yellowish crude glycolide.

The crude glycolide was washed using a 0.1 wt. % alkali solution and deionized water to be neutral, and then vacuum dried at 20° C. for 24 hours. The obtained glycolide has a yield of 98.4% and a purity of 99.9%.

### Example 7

150 kg of a 70 wt. % glycolic acid aqueous solution was added to a reactor for dehydrative oligomerization. First, the glycolic acid aqueous solution was heated from room temperature to 110° C. at a heating rate of 1.5° C./min, and then dehydrated under atmosphere for 1.5 hour. Second, the glycolic acid aqueous solution was heated from 110° C. to 150° C. at a heating rate of 1.5° C./min, and then dehydrated under atmosphere for 1.5 hour. Third, the glycolic acid aqueous solution was further heated from 150° C. to 165° C. at a heating rate of 1.5° C./min, and then dehydrated under atmosphere for 1.5 hour. Fourth, the glycolic acid aqueous solution was further heated from 165° C. to 210° C. at a heating rate of 1.5° C./min, and the absolute pressure of the glycolic acid aqueous solution was reduced from atmosphere to 95.0 torr at a decompression rate of 1.5 torr/min. Hold the temperature and the pressure for 1.5 hour. Thereafter, the absolute pressure of the glycolic acid aqueous solution was reduced from 95.0 torr to 20.0 torr at a decompression rate of 1.5 torr/min for 2.0 hour. A product of oligoglycolic acid having a weight-average molecular weight (M_{w}) of 6.0×10³ was obtained, and a yield thereof was 100%.

150.0 g of benzimidazole and 75.0 g of glycocyamine were added to the product of oligoglycolic acid. The absolute pressure of the reactant mixture was reduced from atmosphere to 5.0 torr at a decompression rate of 1.5 torr/min. Under the pressure, the reactant mixture was heated to 260° C. at a heating rate of 15° C./min, and the temperature was held for 30 minutes. Thereafter, the reactant mixture was heated from 260° C. to 275° C. at a heating rate of 1.5° C./min, and the temperature was held for 30 minutes. Next, the reactant mixture was further heated from 275° C. to 285° C. at a heating rate of 1.5° C./min, and the temperature was held for 70 minutes, to yield a white or yellowish crude glycolide.

The crude glycolide was washed using a 0.1 wt. % alkali solution and deionized water to be neutral, and then vacuum dried at 20° C. for 24 hours. The obtained glycolide has a yield of 98.1% and a purity of 99.9%.

### Example 8

150 kg of a 70 wt. % glycolic acid aqueous solution was added to a reactor for dehydrative oligomerization. First, the glycolic acid aqueous solution was heated from room temperature to 110° C. at a heating rate of 1.5° C./min, and then dehydrated under atmosphere for 1.5 hour. Second, the glycolic acid aqueous solution was heated from 110° C. to 150° C. at a heating rate of 1.5° C./min, and then dehydrated under atmosphere for 1.5 hour. Third, the glycolic acid aqueous solution was further heated from 150° C. to 165° C. at a heating rate of 1.5° C./min, and then dehydrated under atmosphere for 1.5 hour. Fourth, the glycolic acid aqueous solution was further heated from 165° C. to 210° C. at a heating rate of 1.5° C./min, and the absolute pressure of the glycolic acid aqueous solution was reduced from atmosphere to 95.0 torr at a decompression rate of 1.5 torr/min. Hold the temperature and the pressure for 1.5 hour. Thereafter, the absolute pressure of the glycolic acid aqueous solution was reduced from 95.0 torr to 20.0 torr at a decompression rate of 1.5 torr/min for 2.0 hour. A product of oligoglycolic acid having a weight-average molecular weight (M_{w}) of 6.0×10³ was obtained, and a yield thereof was 100%.

105.0 g of benzimidazole and 105.0 g of creatine were added to the product of oligoglycolic acid. The absolute pressure of the reactant mixture was reduced from atmosphere to 5.0 torr at a decompression rate of 1.5 torr/min. Under the pressure, the reactant mixture was heated to 260° C. at a heating rate of 15° C./min, and the temperature was held for 30 minutes. Thereafter, the reactant mixture was heated from 260° C. to 275° C. at a heating rate of 1.5° C./min, and the temperature was held for 30 minutes. Next, the reactant mixture was further heated from 275° C. to 285° C. at a heating rate of 1.5° C./min, and the temperature was held for 70 minutes, to yield a white or yellowish crude glycolide.

The crude glycolide was washed using a 0.1 wt. % alkali solution and deionized water to be neutral, and then vacuum dried at 20° C. for 24 hours. The obtained glycolide has a yield of 99.0% and a purity of 99.9%.

### Example 9

150 kg of a 70 wt. % glycolic acid aqueous solution was added to a reactor for dehydrative oligomerization. First, the glycolic acid aqueous solution was heated from room temperature to 110° C. at a heating rate of 1.5° C./min, and then dehydrated under atmosphere for 1.5 hour. Second, the glycolic acid aqueous solution was heated from 110° C. to 150° C. at a heating rate of 1.5° C./min, and then dehydrated under atmosphere for 1.5 hour. Third, the glycolic acid aqueous solution was further heated from 150° C. to 165° C. at a heating rate of 1.5° C./min, and then dehydrated under atmosphere for 1.5 hour. Fourth, the glycolic acid aqueous solution was further heated from 165° C. to 210° C. at a heating rate of 1.5° C./min, and the absolute pressure of the glycolic acid aqueous solution was reduced from atmosphere to 95.0 torr at a decompression rate of 1.5 torr/min. Hold the temperature and the pressure for 1.5 hour. Thereafter, the absolute pressure of the glycolic acid aqueous solution was reduced from 95.0 torr to 20.0 torr at a decompression rate of 1.5 torr/min for 2.0 hour. A product of oligoglycolic acid having a weight-average molecular weight (M_{w}) of 6.0×10³ was obtained, and a yield thereof was 100%.

75.0 g of benzimidazole and 150.0 g of phosphocreatine were added to the product of oligoglycolic acid. The absolute pressure of the reactant mixture was reduced from atmosphere to 5.0 torr at a decompression rate of 1.5 torr/min. Under the pressure, the reactant mixture was heated to 260° C. at a heating rate of 15° C./min, and the temperature was held for 30 minutes. Thereafter, the reactant mixture was heated from 260° C. to 275° C. at a heating rate of 1.5° C./min, and the temperature was held for 30 minutes. Next, the reactant mixture was further heated from 275° C. to 285° C. at a heating rate of 1.5° C./min, and the temperature was held for 70 minutes, to yield a white or yellowish crude glycolide.

The crude glycolide was washed using a 0.1 wt. % alkali solution and deionized water to be neutral, and then vacuum dried at 20° C. for 24 hours. The obtained glycolide has a yield of 99.2% and a purity of 99.9%.

It will be obvious to those skilled in the art that changes and modifications may be made, and therefore, the aim in the appended claims is to cover all such changes and modifications.

What is claimed is:

1. A method, comprising:
a1) adding a 70 wt. % glycolic acid aqueous solution to a reactor, heating the glycolic acid aqueous solution to a temperature of between 100 and 110° C. at a heating rate of 1.0-1.5° C./min, and maintaining the temperature under atmosphere for 1.0-2.0 hours;
b1) heating the glycolic acid aqueous solution from the temperature of between 100 and 110° C. to a temperature of between 140 and 150° C. at a heating rate of 1.0-1.5° C./min, and maintaining the temperature under atmosphere for 1.0-2.0 hours;
c1) heating the glycolic acid aqueous solution from the temperature of between 140 and 150° C. to a temperature of between 155 and 165° C. at a heating rate of 1.0-1.5° C./min, and maintaining the temperature under atmosphere for 1.0-2.0 hours;
d1) heating the glycolic acid aqueous solution from the temperature of between 155 and 165° C. to a temperature of between 200 and 210° C. at a heating rate of 1.0-1.5° C./min, decreasing a pressure of the glycolic acid aqueous solution from the atmosphere to an absolute pressure of between 90.0 and 95.0 torr at a decompression rate of 1.0-1.5 torr/min, and maintaining the temperature and pressure for 1.0-1.5 hours;
e1) under the temperature of between 200 and 210° C., decreasing the pressure of the glycolic acid aqueous solution from the absolute pressure of between 90.0 and 95.0 torr to an absolute pressure of between 10.0 and 20.0 torr at a decompression rate of 1.0-1.5 torr/min, and maintaining the temperature and pressure for 1.0-2.0 hours, to yield an oligoglycolic acid;
a2) adding a binary complex catalytic system to the oligoglycolic acid obtained in e1), to yield a reactant mixture;
b2) reducing an absolute pressure of the reactant mixture from atmosphere to between 3.0 and 5.0 torr at a decompression rate of 1.0-1.5 torr/min;
c2) heating the reactant mixture to a temperature of between 250 and 260° C. at a heating rate of 10-15° C./min, and maintaining the temperature for 20-30 minutes;
d2) heating the reactant mixture from the temperature of between 250 and 260° C. to a temperature of between 270 and 275° C. at a heating rate of 1.0-1.5° C./min, and maintaining the temperature for 20-30 minutes; and
e2) heating the reactant mixture from the temperature of between 270 and 275° C. to a temperature of between 280 and 285° C. at a heating rate of 1.0-1.5° C./min, and maintaining the temperature for 60-70 minutes, to yield a crude glycolide.

2. The method of claim 1, wherein the binary complex catalytic system comprises a cyclic guanidine compound and a linear guanidine compound; the cyclic guanidine compound is selected from bicyclic guanidine, guanine, and benzimidazole; the linear guanidine compound is selected from glycocyamine, creatine, and phosphocreatine, and an addition amount of each of the cyclic guanidine compound and the linear guanidine compound accounts for 0.05-0.10 wt. % of that of the oligoglycolic acid.

3. The method of claim 1, wherein the oligoglycolic acid obtained in e1) has a weight-average molecular weight (M_{w}) of 5.0-6.0×10³.

4. The method of claim 1, wherein the crude glycolide obtained in e2) is white or yellowish and has a purity greater than or equal to 99.9%.
